(19) United States
(12) Patent Application Publication
(10) Pub. No.: US 2025/0269096 A1
(43) Pub. Date: Aug. 28, 2025

(54) FLUOROPOLYMER MEDICAL DEVICES

(71) Applicant: Unomedical A/S, Lejre (DK)

(72) Inventors: Jeffrey Cafmeyer, Columbus, OH (US); Daniel Garbark, Columbus, OH (US); Rachel Zimet Pytel, Las Vegas, NV (US); Lisa Jaunet van Kooten, Lejre (DK)

(21) Appl. No.: 19/072,921

(22) Filed: Mar. 6, 2025

Related U.S. Application Data
(63) Continuation of application No. PCT/EP2023/073835, filed on Aug. 30, 2023.
(60) Provisional application No. 63/404,253, filed on Sep. 7, 2022.

(30) Foreign Application Priority Data
Oct. 20, 2022 (GB) 2215511.3

(51) Int. Cl. A61L 31/10; A61M 5/158

(52) U.S. Cl. CPC A61L 31/10; A61M 5/158; A61L 2420/02; A61M 2207/00

(57) ABSTRACT

The invention provides a method of activating a fluoropolymer surface of a medical device, the method comprising the steps of: a. Providing a medical device comprising a fluoropolymer surface; and b. Treating the fluoropolymer surface with at least one reducing agent.

### TECHNICAL FIELD OF THE INVENTION

The present invention relates to methods of activating a fluoropolymer surface of a medical device and to methods of functionalising a fluoropolymer surface of a medical device.

### BACKGROUND TO THE INVENTION

Cannulas and catheters are indispensable in the medical field and are inserted into the body, often for the delivery or removal of fluid. The material and configuration of such medical devices vary enormously depending on their intended use. Typical uses of cannulas and catheters include cardiovascular, urological, gastrointestinal, neurovascular, and ophthalmic applications.

There has been recent interest in constructing such medical devices using fluoropolymer materials, in particular polytetrafluoroethylene (PTFE). Such fluoropolymers are advantageous for use in medical applications due to their favourable mechanical properties and excellent chemical stability under biological conditions.

It is often desirable or even necessary to chemically modify the fluoropolymer base materials used to construct such medical devices. This may, for instance, be necessitated due to foreign body responses which can occur when such insertable medical devices are introduced into the body—i.e. where a patient's body identifies the medical device as foreign and rejects it. Such responses can begin as early as on insertion of the medical device into the body, which can cause inflammation and trigger an immediate rush of inflammatory-mediating cells and proteins to the area of insertion. Proteins typically then non-specifically adsorb to the medical device surface, forming a protein layer which becomes a provisional matrix, through which cells and bacteria gathering in the area can identify and interact with the foreign body. Foreign body responses can ultimately cause numerous problems, including device clogging and infection. The negative impacts are often exacerbated when such medical devices are inserted by untrained personnel—e.g. by a user themselves in the absence of a medical professional. Furthermore, acute responses are particularly common for medical devices which are inserted subcutaneously or intravenously into the body, and these can have many harmful and even life-threatening consequences.

Fluoropolymers are chemically inert and notoriously difficult to chemically modify or even coat with a chemical species. Such polymers also display practical incompatibility with a vast range of chemistry commonly employed in medical device surface coatings and additives.

The use of harsh chemistries is commonly required to successfully modify fluoropolymers. However, methods for modifying fluoropolymer medical devices must not only allow for effective chemical modification, but must also result in final products which are safe for medical application.

There exists a need for methods of modifying fluoropolymer medical devices which ameliorate one or more of the above issues.

It is an aim of embodiments of the present invention to address or ameliorate one or more of the above problems of the prior art. In particular, it is an aim of embodiments of the present invention to provide methods which have one or more of the following advantages:
Do not negatively impact, and preferably improve fluoropolymer mechanical properties.
Retain fluoropolymer non-stick and/or lubricious surface properties. Provide medical devices which are easy to insert and remove from the body.
Provide effective modification of the fluoropolymer medical device.
Provide medical devices which are safe for medical application. No chemical leaching from the medical devices, especially when inserted into the body.
High efficiency.
Ease of performance.

It is also an aim of embodiments of the present invention to overcome or mitigate at least one problem of the prior art, whether expressly described herein or not.

### SUMMARY OF THE INVENTION

According to a first aspect of the invention, there is provided a method of activating a fluoropolymer surface of a medical device, the method comprising the steps of:
(a) Providing a medical device comprising a fluoropolymer surface; and
(b) Treating the fluoropolymer surface with at least one reducing agent.

Such a method is particularly effective at producing a highly reactive fluoropolymer surface which can be easily functionalised with chemical species. However, it is not believed that the method has any long-term implications on the stability of the modified surface—the method may in fact aid stability of the modified surface through surface crosslinking interactions generated on treatment with a reducing agent.

In some embodiments, the fluoropolymer is independently chosen from: polytetrafluoroethylene (PTFE), polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof.

In some embodiments, the fluoropolymer is independently selected from the group consisting of: polytetrafluoroethylene (PTFE), polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof.

The fluoropolymer may be independently chosen from: PTFE, fluorinated ethylene-propylene, polyvinylidene fluoride, and combinations, blends or copolymers thereof.

The fluoropolymer may be independently selected from the group consisting of: PTFE, fluorinated ethylene-propylene, polyvinylidene fluoride, and combinations, blends or copolymers thereof.

In a particularly preferred embodiment, the fluoropolymer is or comprises PTFE. PTFE provides excellent mechanical properties.

In some embodiments, the medical device comprises a tubular body comprising the fluoropolymer surface.

The fluoropolymer surface may be or comprise an outer and/or an inner surface of the tubular body. The fluoropolymer surface may preferably be or comprise an outer surface of the tubular body.

In some embodiments, the fluoropolymer surface comprises at least 5% of the outer surface area of the tubular body, or at least 10, 20, 30, 40, 50, 60, or preferably at least 70, or at least 80, 90, 95, 96, 97, 98, or at least 99% of the outer surface area of the tubular body, or 100% of the outer surface area of the tubular body. The fluoropolymer surface may comprise no greater than 95%, or no greater than 90, 85, or no greater than 80% of the outer surface area of the tubular body.

In preferred embodiments, the medical device is an insertable medical device. In some embodiments, the medical device is a cannula or a catheter, preferably which is configured to be inserted into a body.

In some embodiments, the cannula or catheter is independently chosen from: a urinary cannula or catheter, an intravenous cannula or catheter, a nasal cannula or catheter, and a microcannula.

In some embodiments, the cannula or catheter is independently selected from the group consisting of: a urinary cannula or catheter, an intravenous cannula or catheter, a nasal cannula or catheter, and a microcannula.

The cannula or catheter may be an indwelling (Foley) catheter or cannula. Such a cannula/catheter is typically inserted and kept in a body for long periods of time, such as several days to months. Alternatively, the cannula or catheter may be an intermittent catheter or cannula. Such a cannula/catheter is typically inserted into a body for short time periods, such as less than a day.

In preferred embodiments, the medical device is a cannula that is part of an infusion set. The cannula may be part of an infusion set comprising a body which comprises a fluid part. In some embodiments, the body of the infusion set is attachable to the body of a user, in use. The body of the infusion set may be attachable to the body of the user via an adhesive part, in use. The adhesive part may be attachable to skin, in use. The adhesive part may attach the body of the infusion set to the user's skin, in use. The fluid part may be connected to the body of the infusion set or comprise part of the body of the infusion set. The fluid part may provide a fluid path through the infusion set. The fluid part may allow for fluid communication between the body of the infusion set and the cannula. The cannula may be attached to the fluid part or directly to the body of the infusion set. An end of the cannula may preferably be insertable into the body of a user, in use. In some embodiments, the cannula comprises an insertion needle on an end thereof, which can help to insert the cannula into the body of the user. The infusion set may further comprise an inserter part to assist insertion of the cannula into the body of the user. The inserter part may be an automatic inserter part or a manual inserter part.

The infusion set may further comprise a pump. The pump may assist in transporting substances from the infusion set into the body of a user, and vice versa. In some embodiments, the pump is attached to the insertion set via a connector. The pump may be attached to the body of the infusion set via the connector. The connector may comprise a tube which may be attached to a hub which controls the pump.

In some embodiments, the medical device is a cannula that is part of a patch pump. The patch pump may comprise a patch that is attachable to the body of a user, in use. The patch may comprise an adhesive. The patch may be attachable to skin through the adhesive, in use. The patch may comprise a fluid part. The fluid part may provide a fluid path through the patch pump. The cannula may be attached to the fluid part. An end of the cannula may preferably be insertable into the body of a user, in use. In some embodiments, the cannula comprises an insertion needle on an end thereof, which can help to insert the cannula into the body of the user. The patch may further comprise a pump, which may be an integral part of the patch or may be attached thereto. The pump may assist in transporting substances from the patch pump into the body of a user, and vice versa.

In some preferred embodiments, the method of activating a fluoropolymer surface of a medical device comprises the steps of:
(a) Providing a cannula that is part of an infusion set or patch pump and which comprises a fluoropolymer surface; and
(b) Treating the fluoropolymer surface with at least one reducing agent.

In some embodiments, the cannula is part of an infusion set or patch pump for the delivery of a substance into the body. The cannula may be part of an intravenous and/or subcutaneous infusion set or patch pump. The cannula may be part of an infusion set or patch pump for the subcutaneous delivery of a substance into the body, such as for the subcutaneous delivery on insulin into the body.

In some embodiments, the catheter or cannula comprises a hollow tubular body. The hollow tubular body may comprise an outer surface and/or an inner surface. The outer surface may comprise at least one chosen from: an external facing surface of the body, a lumen of the body, and any eyelets present on the body. The outer surface may comprise at least one of the group consisting of: an external facing surface of the body, a lumen of the body, and any eyelets present on the body. In preferred embodiments, the outer surface is the external-facing surface of the body and/or the inner lumen. In some embodiments, the outer surface may comprise the external-facing surface of the body, the inner lumen, and the eyelets. The inner surface of the body may comprise a lumen of the body.

In some embodiments, step (a) comprises forming the medical device by a melt-extrusion or injection moulding procedure. The method may comprise melt-extruding or injection moulding a fluoropolymer to form a tubular body of the medical device. In some embodiments, the fluoropolymer is provided in granulate or powder form prior to melt-extrusion or injection-moulding.

In some embodiments, step (b) comprises introducing at least one reactive group on the fluoropolymer surface. Step (b) may comprise cleaving at least one polymer chain on the fluoropolymer surface, and introducing at least one reactive group on the surface. In some embodiments, at least one reactive group comprises at least one electronegative atom. In some embodiments, at least one reactive group may be independently chosen from: an oxygen-containing moiety, an unsaturated moiety, a radical, and combinations thereof. In some embodiments, at least one reactive group may be independently selected from the group consisting of: an oxygen-containing moiety, an unsaturated moiety, a radical, and combinations thereof.

Step (b) may comprise reducing the fluoropolymer surface and then oxidising said surface. In some embodiments, step (b) or part of step (b) is performed under atmospheric oxygen conditions. In other embodiments, step (b) or part of step (b) is performed under an oxygen enriched atmosphere. In some embodiments, step (b) is performed under an oxygen enriched atmosphere after treatment of the fluoropolymer surface with at least one reducing agent. Step (b) may produce an activated fluoropolymer surface comprising at least one oxygen-containing reactive moiety. At least one oxygen-containing moiety may be independently chosen from: a peroxy group, a hydroxy group, a carbonyl group, and derivatives and/or combinations thereof. At least one oxygen-containing moiety may be independently selected from the group consisting of: a peroxy group, a hydroxy group, a carbonyl group, and derivatives and/or combinations thereof. The carbonyl group may be independently chosen from: a carboxyl group, an aldehyde, a ketone, an acid fluoride, and combinations thereof. The carbonyl group may be independently selected from the group consisting of: a carboxyl group, an aldehyde, a ketone, an acid fluoride, and combinations thereof.

In some preferred embodiments, the method of functionalising a fluoropolymer surface of a medical device comprises the steps of:
(a) Providing a medical device comprising a fluoropolymer surface;
(b) Treating the fluoropolymer surface with at least one reducing agent and then oxidising the surface; and
(c) Functionalising the oxidised surface with at least one chemical species.

In some embodiments, step (b) comprises producing an activated fluoropolymer surface comprising at least one unsaturated reactive moiety. At least one unsaturated reactive moiety may be independently chosen from: an alkene, an alkyne, and derivatives and/or combinations thereof. At least one unsaturated reactive moiety may be independently selected from the group consisting of: an alkene, an alkyne, and derivatives and/or combinations thereof. Such unsaturated reactive moieties may react via polymerisation-type reactions. Polymerisation-type reactions may involve any suitable polymerisation process, such as conventional condensation, addition or free radical graft polymerization (FRGP) or controlled radical polymerization (CRP), such as ATRGP, RAFT and NMGP.

Step (b) preferably comprises activating the fluoropolymer surface. In some embodiments, step (b) comprises the step of activating the fluoropolymer surface across at least 5% of the total area of the fluoropolymer surface, or at least 10, 20, 30, 40, 50, 60, 70, 80, 90, 95, 96, 97, 98, or across at least 99% of the total area of the fluoropolymer surface, or 100% of the total area of the fluoropolymer surface. Step (b) may comprise the step of activating the fluoropolymer surface across no greater than 95% of the total area of the fluoropolymer surface, or across no greater than 90, 85, or no greater than 80% of the total area of the fluoropolymer surface.

Step (b) may comprise defluorinating or partially defluorinating the fluoropolymer surface. Step (b) may comprise defluorinating at least 5% of the fluoropolymer surface, or at least 10, 20, 30, 40, 50, 60, 70, 80, 90, 95, 96, 97, 98, or at least 99% of the fluoropolymer surface, or 100% of the fluoropolymer surface. Step (b) may comprise defluorinating no greater than 95% of the fluoropolymer surface, or no greater than 90, 85, or no greater than 80% of the fluoropolymer surface.

Step (b) may comprise reducing the average fluorine-to-carbon atomic ratio (F/C ratio) of the fluoropolymer surface to a value of no greater than 1.2, or no greater than 1, 0.9, 0.8, 0.7, 0.6, 0.5, 0.4, 0.3, or preferably no greater than 0.2, or no greater than 0.1.

In some preferred embodiments, the method of activating a fluoropolymer surface of a medical device comprises the steps of:
(a) Providing a medical device comprising a fluoropolymer surface; and
(b) Treating the fluoropolymer surface with at least one reducing agent, such that the average fluorine-to-carbon atomic ratio of the fluoropolymer surface is reduced to a value of no greater than 0.3, preferably no greater than 0.2.

Step (b) may comprise increasing the average surface energy of the fluoropolymer surface to a value of at least 25 mN/m, or at least 30, 35, 40, 45, or preferably at least 50, 55, 60, or at least 65 mN/m.

In some preferred embodiments, the method of activating a fluoropolymer surface of a medical device comprises the steps of:
(a) Providing a medical device comprising a fluoropolymer surface; and
(b) Treating the fluoropolymer surface with at least one reducing agent, such that the average surface energy of the fluoropolymer surface is increased to a value of at least 25 mN/m.

Step (b) may comprise reducing the average contact angle of the fluoropolymer surface to a value of no greater than 80°, or no greater than 70, 60, 50, 40, or no greater than 30°.

In some preferred embodiments, the method of activating a fluoropolymer surface of a medical device comprises the steps of:
(a) Providing a medical device comprising a fluoropolymer surface; and
(b) Treating the fluoropolymer surface with at least one reducing agent, such that the average contact angle of the fluoropolymer surface is reduced to a value of no greater than 80°.

In some embodiments, the reducing agent may act to transfer electrons to the fluoropolymer surface.

Treating the fluoropolymer surface with at least one reducing agent may generate at least one surface reactive group. In some embodiments, at least one reactive group may be as described in statements above and may be independently chosen from: an oxygen-containing moiety, an unsaturated moiety, a radical, and combinations thereof. In some embodiments, at least one reactive group may be as described in statements above and may be independently selected from the group consisting of: an oxygen-containing moiety, an unsaturated moiety, a radical, and combinations thereof. In some embodiments, treating the fluoropolymer surface with at least one reducing agent generates at least one surface reactive group independently selected from: an unsaturated, a radical, and combinations thereof; and said surface reactive group further reacts to generate at least one oxygen-containing moiety. In such embodiments, the surface reactive group may react with atmospheric oxygen to generate at least one oxygen-containing moiety. At least one oxygen-containing moiety may be as described in statements of invention above.

Step (b) may comprise the steps of: transferring at least one electron from the reducing agent to the fluoropolymer surface to generate a negatively charged or partially negatively charged surface group; and removing at least one fluorine from the surface group to generate a neutral defluorinated surface group. Fluorine may be removed from the surface group as fluoride or a derivative thereof. In some embodiments, the above steps may produce a radical-containing neutral defluorinated surface group. The above steps may be repeated to generate a non-radical neutral defluorinated surface group. The non-radical neutral defluorinated surface group may comprise a reactive group, preferably an unsaturated moiety, such as an alkene.

At least one reducing agent used in step (b) of the invention may be independently chosen from: an alkali metal, an alkaline earth metal, a group III metal, a transition metal, and combinations thereof.

At least one reducing agent used in step (b) of the invention may be independently selected from the group consisting of: an alkali metal, an alkaline earth metal, a group III metal, a transition metal, and combinations thereof.

In preferred embodiments, at least one reducing agent comprises an alkali metal and/or an alkaline earth metal. At least one reducing agent may preferably comprise an alkali metal. At least one reducing agent may comprise an alkali metal independently chosen from: lithium, potassium, sodium, and combinations thereof. At least one reducing agent may comprise an alkali metal independently selected from the group consisting of: lithium, potassium, sodium, and combinations thereof. In a particularly preferred embodiment, at least one reducing agent comprises sodium.

In some embodiments, step (b) may comprise treating the fluoropolymer surface with at least one reducing agent in the presence of a stabilising species. The stabilising species may complex the reducing agent, preferably in the form of a salt. The stabilising species may accept an electron from the reducing agent, preferably to form a radical anion. The stabilising species may preferably be aromatic, preferably an aromatic compound. The stabilising species may be a polycyclic aromatic compound. The stabilising species may be independently chosen from: benzene, naphthalene, biphenyl, anthracene, pyrene, acenaphthylene, perylene, and derivatives thereof. The stabilising species may be independently selected from the group consisting of: benzene, naphthalene, biphenyl, anthracene, pyrene, acenaphthylene, perylene, and derivatives thereof. The stabilising species may preferably be naphthalene or a derivative thereof. In preferred embodiments, at least one reducing agent comprises an alkali metal and a naphthalene stabilising species which forms an alkali metal naphthalide, preferably sodium naphthalide.

At least one reducing agent may be provided as a solution. At least one reducing agent may be dissolved in a carrier solvent to provide the solution. The carrier solvent may comprise an aprotic solvent. The carrier solvent may comprise an ether, preferably an aprotic ether. In some embodiments, the carrier solvent comprises a glycol ether, preferably an aprotic glycol ether, such as a dialkyl glycol ether. In preferred embodiments, the carrier solvent is independently chosen from: monoglyme, diglyme, tetraglyme, and combinations thereof. In preferred embodiments, the carrier solvent is independently selected from the group consisting of: monoglyme, diglyme, tetraglyme, and combinations thereof. In a particularly preferred embodiment, the carrier solvent comprises diglyme.

Particularly preferably, the reducing agent comprises an alkali metal, preferably sodium and the carrier solvent comprises an aprotic glycol ether, preferably a dialkyl glycol ether, more preferably diglyme.

Such solvents enable high temperature etching, which accelerates and reduces the length of the surface treatment process.

In some embodiments, at least one reducing agent is provided as a solution and the method further comprises the step of preheating the solution before treating the fluoropolymer surface with said solution. The method may comprise preheating the solution at a temperature of at least 30° C., or at least 35, 40, 45, 50, 55, or at least 60° C. The method may comprises preheating the solution at a temperature of no greater than 300° C., or no greater than 250, 200, 150, or no greater than 100° C. The method may comprise preheating the solution at a temperature of between 30-90° C., or between 40-80, 50-70, or between 55-65° C. The preheating step may be performed for at least 5 minutes, or at least 10, 15, 20, 25, 30, 35, 40, 45, 50, 55, or at least 60 minutes. The preheating step may be performed for no greater than 7 hours, or no greater than 6, 5, 4, 3, 2, or no greater than 1.5 hours. The preheating step may be performed for between 30-90 minutes, or between 40-80, 50-70, or between 55-65 minutes.

In some embodiments, at least one reducing agent is provided as a solution and the method further comprises the step of agitating the solution before treating the fluoropolymer surface with said solution. The agitation step may be performed after a preheating step as described above. The agitation step may be performed for between 1-30 seconds, or between 2-5 seconds.

In some embodiments, step (b) comprises treating the fluoropolymer surface with at least one reducing agent at a temperature of at least 5° C., or at least 10, 15, 20, 25, 30, 35, 40, or at least 45° C. Step (b) may comprise treating the fluoropolymer surface with at least one reducing agent at a temperature of no greater than 500° C., or no greater than 450, 400, 350, 300, 250, 200, 150, 100, 90, 80, 70, 60, or no greater than 50° C. Step (b) may comprise treating the fluoropolymer surface with at least one reducing agent at a temperature of between 5-100° C., or between 10-95, 20-90, 25-85, 30-80, 35-75, 40-70, 45-65, or between 50-65° C. Step (b) may comprise treating the fluoropolymer surface with at least one reducing agent at a temperature of between 10-70° C., or between 15-65, or between 20-60° C.

In some preferred embodiments, the method of activating a fluoropolymer surface of a medical device comprises the steps of:
(a) Providing a medical device comprising a fluoropolymer surface; and
(b) Treating the fluoropolymer surface with at least one reducing agent at a temperature of between 45-65° C., or between 50-65° C.

Such temperatures allow more active reducing agent to be released. Reducing agent viscosity is also reduced which allows for wetting of high aspect ratio features of the medical device.

In some embodiments, step (b) comprises treating the fluoropolymer surface with at least one reducing agent at a temperature of between 30-80° C., or between 35-75, 40-70, 45-65, or between 50-60° C.; and wherein the reducing agent is dissolved in a glycol ether carrier solvent, preferably an aprotic glycol ether solvent, more preferably a dialkyl glycol ether.

Step (b) may comprise treating the fluoropolymer surface with the reducing agent for at least 1 second, or at least 2, 3, 4, 5, 10, 15, 20, 25, or at least 30 seconds. Step (b) may comprise treating the fluoropolymer surface with the reducing agent for no greater than 300 seconds, or no greater than 280, 260, 240, 220, 200, 180, 160, 140, 120, 100, 80, or no greater than 60 seconds. Step (b) may comprise treating the fluoropolymer surface with the reducing agent for between 5-180 seconds, or between 10-160, 15-140, 20-120, 25-110, 30-100, 35-90, 40-80, 50-70, or between 55-65 seconds. Step (b) may comprise treating the fluoropolymer surface with the reducing agent for between 5-55 seconds, or between 10-50, 15-45, 20-40, or between 25-35 seconds.

Step (b) may comprise applying the reducing agent to the fluoropolymer surface, preferably as a solution. Step (b) may comprise submerging the medical device or the fluoropolymer surface in the solution.

In some embodiments, the method further comprises a further step of sonicating the fluoropolymer surface. The sonication step may be performed after step (b). The sonication step may be performed for between 1-30 minutes, or between 5-20 minutes, or between 5-15 minutes. The sonication step may be performed in a polar solvent, which may be a polar protic solvent. The solvent may be an aqueous solvent and may be water.

The method may comprise a further step of washing the fluoropolymer surface. The washing step may be performed after step (b). The fluoropolymer surface may be washed with a solvent, which may be a polar solvent. The solvent may be a polar protic solvent. The solvent may comprise an alcohol and/or water. The washing step may be performed at a temperature of between 20-120° C., or between 40-100, or between 60-80° C. The washing step may comprise a first washing step at ambient temperature and a second washing step at a temperature range independently selected from the above range. The first washing step may be performed with an organic solvent, preferably a polar organic solvent. The polar organic solvent may comprise a polar protic solvent, such as an alcohol. The second step may be performed with an aqueous solution or with water, preferably with deionised water.

In some embodiments, the method further comprises the step of treating the fluoropolymer surface with ultraviolet light.

In some preferred embodiments, the method of activating a fluoropolymer surface of a medical device comprises the steps of:
(a) Providing a medical device comprising a fluoropolymer surface; and
(b) Treating the fluoropolymer surface with at least one reducing agent; and wherein the method further comprises the step of treating the fluoropolymer surface with ultraviolet light.

The ultraviolet treating step may be performed before or after step (b). In some embodiments, the method comprises treating the fluoropolymer surface with ultraviolet light having a wavelength of between 100-400 nm, or between 200-400, or between 250-400 nm, or between 300-400, or between 350-400 nm. The method may comprise treating the fluoropolymer surface with ultraviolet light having a wavelength of between 100-350 nm, or between 100-300, or between 100-250 nm.

According to a second aspect of the invention, there is provided a method of functionalising a fluoropolymer surface of a medical device, the method comprising the steps of:
(a) Providing a medical device comprising a fluoropolymer surface;
(b) Treating the fluoropolymer surface with at least one reducing agent; and
(c) Functionalising the treated surface with at least one chemical species.

The fluoropolymer surface and/or medical device are preferably the fluoropolymer surface and medical device of the first aspect of the invention.

Steps (a) and (b) of the method of the second aspect of the invention are preferably steps (a) and (b) of the method of the first aspect of the invention.

Statements of invention above relating to the first aspect of the invention may also be applied mutatis mutandis to the second aspect of the invention. Statements of invention below relating to the second aspect of the invention may also be applied mutatis mutandis to the first aspect of the invention.

Step (c) may comprise applying at least one species to the treated surface as a coating comprising the species. Step (c) may comprise applying at least one species as a coating to at least 5% of the total surface area of the treated surface, or at least 10, 15, 20, 25, 30, 35, 40, 45, 50, 55, 60, 65, 70, 75, 80, 85, 90, 95, 96, 97, 98, or at least 99% of the total surface area of the treated surface, preferably at least 75% or at least 90% of the total surface area of the treated surface or between 75% and 100% of the total surface area of the treated surface. In some embodiments, step (c) comprises applying at least one species as a coating to no greater than 95% of the total surface area of the treated surface, or no greater than 90, 85, or no greater than 80% of the total surface area of the treated surface.

In some embodiments, at least 75% of the coating is the species, or at least 76, 77, 78, 79, 80, 81, 82, 83, 84, 85, 86, 87, 88, 89, 90, 91, 92, 93, 94, 95, 96, 97, 98, 99, or 100% of the coating is the species. In some embodiments, no greater than 95, 90, 85, or no greater than 80% of the coating is the species.

In some embodiments, step (c) comprises adsorbing at least one chemical species to the treated surface. In some embodiments, step (c) comprises covalently bonding at least one chemical species to the surface. Step (c) may comprise ionically or electrostatically bonding at least one chemical species to the surface.

Step (c) may comprise adsorbing at least one species to at least 5% of the total surface area of the treated surface, or at least 10, 15, 20, 25, 30, 35, 40, 45, 50, 55, 60, 65, 70, 75, 80, 85, 90, 95, 96, 97, 98, or at least 99% of the total surface area of the treated surface, preferably at least 75% or at least 90% of the total surface area of the treated surface or between 75% and 100% of the total surface area of the treated surface. In some embodiments, step (c) comprises adsorbing at least one species to no greater than 95% of the total surface area of the treated surface, or no greater than 90, 85, or no greater than 80% of the total surface area of the treated surface.

In some embodiments, step (c) comprises functionalising the treated surface with at least one polymer. At least one polymer may be a homopolymer and/or copolymer.

Step (c) may comprise functionalising the treated surface with at least one hydrophilic or hydrophobic polymer.

Step (c) may comprise functionalising the treated surface with at least one polymer independently chosen from: a linear polymer, a branched polymer, a dendritic polymer, a star polymer, a dendronized polymer, a comb polymer, a polymer brush, a ladder polymer, and combinations thereof.

Step (c) may comprise functionalising the treated surface with at least one polymer independently selected from the group consisting of: a linear polymer, a branched polymer, a dendritic polymer, a star polymer, a dendronized polymer, a comb polymer, a polymer brush, a ladder polymer, and combinations thereof.

In some embodiments, step (c) may comprise treating the surface with at least one species independently chosen from: a polymer, a monomer, and combinations thereof.

In some embodiments, step (c) may comprise treating the surface with at least one species independently selected from the group consisting of: a polymer, a monomer, and combinations thereof.

Step (c) may comprise polymerising at least one polymer from the treated surface. In some embodiments, step (b) comprises creating at least one polymerisation initiation site on the surface; and step (c) comprises polymerising a monomer on at least one polymerisation initiation site to functionalise the surface with at least one polymer.

In some preferred embodiments, the method of functionalising a fluoropolymer surface of a medical device comprises the steps of:
(a) Providing a medical device comprising a fluoropolymer surface;
(b) Treating the fluoropolymer surface with at least one reducing agent to create at least one polymerisation initiation site on the surface; and
(c) Polymerising a monomer on at least one polymerisation initiation site to functionalise the treated surface with at least one polymer.

Step (c) may comprise grafting at least one polymer to the treated surface. In other embodiments, step (b) comprises creating at least one polymerisation initiation site on the surface; and step (c) comprises grafting a polymer to at least one initiation site to functionalise the surface with at least one polymer.

In some embodiments, step (c) comprises polymerising at least one polymeric species from the treated surface to produce a polymer comprising macromonomers.

Step (c) may comprise treating the surface with a solution of at least one chemical species or monomer thereof in a solvent. The solvent may be a polar solvent, which may be a polar protic solvent. The solvent may comprise water. Alternatively, the solvent may comprise an organic solvent, which may be a polar organic solvent. The organic solvent may be independently chosen from: an alcohol, an ether, an ester, a ketone, an aldehyde, an amide, a nitrile, a sulfoxide, a carbonate, a carboxylic acid, and combinations thereof. The organic solvent may be independently selected from the group consisting of: an alcohol, an ether, an ester, a ketone, an aldehyde, an amide, a nitrile, a sulfoxide, a carbonate, a carboxylic acid, and combinations thereof. The chemical species or monomer thereof may be present in the solution at a total concentration of at least 0.05 wt. %, or at least 0.1, 0.2, 0.3, 0.4, 0.5, 0.6, 0.7, 0.8, 0.9, or at least 1 wt. %. The chemical species or monomer thereof may be present in the solution at a total concentration of no greater than 10 wt. %, or no greater than 9, 8, 7, 6, 5, 4, 3, 2, or no greater than 1 wt. %. The chemical species or monomer thereof may be present in the solution at a total concentration of between 0.05-5 wt %, or between 0.1-2 wt. %, or between 0.5-1.5, or between 0.75-1.25 wt. %.

In some embodiments, step (c) comprises hydrophilizing the surface. Step (c) may comprise reacting the treated surface with a hydrophilic functional group-containing polymer or monomer. The hydrophilic polymer or monomer may comprise a polymer or monomer units of a polymer independently chosen from: an ethylenically-unsaturated polymer with hydrophilic, charged or polar functional groups; a polyalkylene glycol polymer; an acrylate or alkacrylate polymer with hydrophilic, charged or polar functional groups; an N-vinyl lactam; and combinations thereof. The hydrophilic polymer or monomer may comprise a polymer or monomer units of a polymer independently selected from the group consisting of: an ethylenically-unsaturated polymer with hydrophilic, charged or polar functional groups; a polyalkylene glycol polymer; an acrylate or alkacrylate polymer with hydrophilic, charged or polar functional groups; an N-vinyl lactam; and combinations thereof.

In some embodiments, step (c) comprises functionalising the surface with at least one polyalkylene glycol. At least one polyalkylene glycol may have at least one monomer unit having between 1-8 carbons in the alkyl chain. At least one polyalkylene glycol may have a polyethylene glycol monomer unit and/or polypropylene glycol monomer unit. At least one polyalkylene glycol polymer or monomer thereof may comprise a reactive end group via which it is bonded to the surface in step (c). The reactive end-group may be an acrylate or alkacrylate group, such as a methacrylate end-group.

The polyalkylene glycol, such as polyethylene glycol or polypropylene glycol, may have a molecular weight (Mw) of no more than 10,000. In some embodiments the Mw of the polyalkylene glycol, such as polyethylene glycol or polypropylene glycol, may have a molecular weight of no more than 5000, 2500, 1500 or no more than 1000. In some embodiments the polyalkylene glycol comprises polyethylene glycol or polypropylene glycol having a Mw of between 100 and 1000, such as between 100 and 800, especially 200-400.

At least one polyalkylene glycol monomer may have any other suitable reactive end group, such as an end group chosen from: epoxy, vinyl, thiol, silane, aldehyde, amine, azide, biotin, carboxylic acid, fluorescent, halide, hydrazide, hydroxyl, lipid, maleimide, norborene, alkyne, olefin, phosphate, pyrene, sulfonate, and vinyl sulfone. At least one polyalkylene glycol monomer may have any other suitable reactive end group, such as an end group selected from the group consisting of epoxy, vinyl, thiol, silane, aldehyde, amine, azide, biotin, carboxylic acid, fluorescent, halide, hydrazide, hydroxyl, lipid, maleimide, norborene, alkyne, olefin, phosphate, pyrene, sulfonate, and vinyl sulfone. Alternatively, at least polyalkylene glycol (PAG) may be chosen from: a 4-arm PAG, 8-arm PAG, amphiphilic PAG, heterobifunctional PAG, homobifunctional PAG, hyperbranched dendrimer PAG, methoxylinear PAG and monodisperse PAG. Alternatively, at least polyalkylene glycol (PAG) may be selected from the group consisting of a 4-arm PAG, 8-arm PAG, amphiphilic PAG, heterobifunctional PAG, homobifunctional PAG, hyperbranched dendrimer PAG, methoxylinear PAG and monodisperse PAG.

In some embodiments, step (c) comprises hydrophobicizing the surface. Step (c) may comprise reacting the treated surface with a hydrophobic functional group-containing polymer or monomer. The hydrophobic polymer or monomer may comprise a polymer or monomer units of a polymer independently chosen from: a hydrophobic acrylate, a hydrophobic alkacrylate, a hydrophobic silane, and combinations thereof. The hydrophobic polymer or monomer may comprise a polymer or monomer units of a polymer independently selected from the group consisting of: a hydrophobic acrylate, a hydrophobic alkacrylate, a hydrophobic silane, and combinations thereof.

In any of the embodiments described herein in which polymerisation is performed, any suitable polymerisation process may be used, such as conventional condensation, addition or free radical graft polymerization (FRGP) or controlled radical polymerization (CRP), such as ATRGP, RAFT and NMGP.

In some embodiments, the fluoropolymer surface is functionalised with at least one chemical species via a free radical polymerisation method. The free radical polymerisation method may comprise a controlled/living free radical polymerisation method. These techniques are known to a skilled person in the art and employ the principle of an equilibrium between free radicals and various types of dormant species (depending on the specific type of polymerisation technique employed).

The controlled/living radical polymerisation techniques include nitroxide-mediated polymerisation, reversible addition fragmentation transfer polymerisation (RAFT) and atom transfer radical polymerisation (ATRP).

More detailed descriptions of polymerisation mechanisms and related chemistry is discussed for nitroxide-mediated polymerisation (Chapter 10, pages 463 to 522), ATRP (Chapter 11, pages 523 to 628) and RAFT (Chapter 12, pages 629 to 690) in the Handbook of Radical Polymerization, edited by Krzysztof Matyjaszewski and Thomas P. Davis, 2002, published by John Wiley and Sons Inc.

The controlled/living polymerisation processes leave a residue of reagent on the polymer chain such as (nitroxyl group from nitroxide-mediated), or a halogen from ATRP, thiocarbonylthio group from RAFT.

In some embodiments it is desirable to remove the residue i.e., remove the nitroxyl, halogen or thiocarbonylthio group. Processes are known to a skilled person to remove such groups, and the disclosure in EP2791184 provides a solution to remove thiocarbonylthio groups. Other such techniques are described, for example, in Chong et at, Macromolecules 2007, 40, 4446-4455; Chong et al, Aust. J. Chem. 2006, 59, 755-762; Postma et al, Macromolecules 2005, 38, 5371-5374; Moad et al, Polymer International 60, no. 1, 2011, 9-25; and Wilcock et al, Polym. Chem., 2010, 1, 149-157.

In ATRP polymerisation, groups that may be transferred by a radical mechanism include halogens (from a halogen-containing compound) or various ligands. A more detailed review of groups that may be transferred is described in U.S. Pat. No. 6,391,996.

Examples of a halogen-containing compound that may be used in ATRP polymerisation include benzyl halides such as p-chloromethylstyrene, α-dichloroxylene, α,α-dichloroxylene, α,α-dibromoxylene, hexakis(α-bromomethyl)benzene, benzyl chloride, benzyl bromide, 1-bromo-1-phenylethane and 1-chloro-1-phenylethane; carboxylic acid derivatives which are halogenated at the α-position, such as propyl 2-bromopropionate, methyl 2-chloropropionate, ethyl 2-chloropropionate, methyl 2-bromopropionate, and ethyl 2-bromoisobutyrate; tosyl halides such as p-toluenesulfonyl chloride; alkyl halides such as tetrachloromethane, tribromomethane, 1-vinylethyl chloride, and 1-vinylethyl bromide; and halogen derivatives of phosphoric acid esters, such as dimethylphosphoric acid.

In one embodiment when the halogen compound is employed, a transition metal such as copper is also present. The transition metal may be in the form of a salt. The transition metal is capable of forming a metal-to-ligand bond and the ratio of ligand to metal depends on the dentate number of the ligand and the co-ordination number of the metal. The ligand may be a nitrogen or phosphorus-containing ligand.

Examples of a suitable ligand include triphenylphosphine, 2,2-bipyridine, alkyl-2,2-bipyridine, such as 4,4-di-(5-heptyl)-2,2-bipyridine, tris(2-aminoethyl)amine (TREN), N,N,N′,N′,N″-pentamethyldiethylenetriamine, 4,4-do-(5-nonyl)-2,2-bipyridine, 1,1,4,7,10,10-hexamethyltriethylenetetramine and/or tetramethylethylenediamine. Further suitable ligands are described in, for example, International Patent application WO 97/47661. The ligands may be used individually or as a mixture. In one embodiment the nitrogen containing ligand is employed in the presence of copper. In one embodiment the ligand is phosphorus-containing with triphenyl phosphine (PPb₃) a common ligand. A suitable transition metal for a triphenyl phosphine ligand includes Rh, Ru, Fe, Re, Ni or Pd.

In RAFT polymerisation, a chain transfer agent may be used, A more detailed review of suitable chain transfer agents RAFT polymerisation, as described in International Patent Publication Nos. WO 98/01478, WO 99/31144 and WO 10/83569, is a polymerisation technique that exhibits characteristics associated with living polymerisation.

Examples of a suitable RAFT chain transfer agent include benzyl 1-(2-pyrrolidinone)carbodithioate, benzyl(1,2-benzenedicarboximido) carbodithioate, 2-cyanoprop-2-yl 1-pyrrolecarbodithioate, 2-cyanobut-2-yl 1-pyrrolecarbodithioate, benzyl 1-imidazolecarbodithioate, N,N-dimethyl-S-(2-cyanoprop-2-yl)dithiocarbamate, N,N-diethyl-S-benzyl dithiocarbamate, cyanomethyl 1-(2-pyrrolidone) carbodithoate, cumyl dithiobenzoate, 2-dodecylsulphanylthiocarbonylsulphanyl-2-methyl-propionic acid butyl ester, O-phenyl-S-benzyl xanthate, N,N-diethyl S-(2-ethoxy-carbonylprop-2-yl)dithiocarbamate, dithiobenzoic acid, 4-chlorodithiobenzoic acid, O-ethyl-S-(1-phenylethyl)xanthtate, O-ethyl-S-(2-(ethoxycarbonyl) prop-2-yl)xanthate, O-ethyl-S-(2-cyanoprop-2-yl)xanthate, O-ethyl-S-(2-cyanoprop-2-yl) xanthate, O-ethyl-S-cyanomethyl xanthate, O-pentafluorophenyl-S-benzyl xanthate, 3-benzylthio-5,5-dimethylcyclohex-2-ene-1-thione or benzyl 3,3-di(benzylthio) prop-2-enedithioate, S,S′-bis-(α,α′-disubstituted-α″-acetic acid)-trithiocarbonate, S,S′-bis-(α,α′-disubstituted-α″-acetic acid)-trithiocarbonate or S-alkyl-S′-(α,α′-disubstituted-α″-acetic acid)-trithiocarbonates, benzyl dithiobenzoate, 1-phenylethyl dithiobenzoate, 2-phenylprop-2-yl dithiobenzoate, 1-acetoxyethyl dithiobenzoate, hexaki(thiobenzoylthiomethyl)benzene, ,4-bis(thiobenzoylthiomethyl)benzene, 1,2,4,5-tetrakis (thiobenzoylthiomethyl)benzene, 1,4-bis-(2-(thiobenzoylthio)-prop-2-yl)benzene, 1-(4-methoxyphenyl) ethyl dithiobenzoate, benzyl dithioacetate, ethoxycarbonylmethyl dithioacetate, 2-(ethoxycarbonyl)prop-2-yl dithiobenzoate, 2,4,4-trimethylpent-2-yl dithiobenzoate, 2-(4-chlorophenyl)prop-2-yl dithiobenzoate, 3-vinylbenzyl dithiobenzoate, 4-vinylbenzyl dithiobenzoate, S-benzyl diethoxyphosphinyldithioformate, tert-butyl trithioperbenzoate, 2-phenylprop-2-yl 4-chlorodithiobenzoate, 2-phenylprop-2-yl 1-dithionaphthalate, 4-cyanopentanoic acid dithiobenzoate, dibenzyl tetrathioterephthalate, dibenzyl trithiocarbonate, carboxymethyl dithiobenzoate or poly(ethylene oxide) with dithiobenzoate end group or mixtures thereof.

In one embodiment a suitable RAFT chain transfer agent includes 2-Dodecylsulfanylthiocarbonylsulfanyl-2-methyl-propionic acid butyl ester, cumyl dithiobenzoate or mixtures thereof.

A discussion of the polymer mechanism of RAFT polymerisation is shown on page 664 to 665 in section 12.4.4 of Matyjaszewski et al.

In some embodiments, the fluoropolymer surface is functionalised with at least one species via an ionic polymerisation method, which may be an anionic polymerisation method. In such embodiments, at least one carboxybetaine and/or sulfobetaine species monomer may be functionalised with an epoxide group; said group being able to participate in an anionic polymerisation process. The ionic polymerisation method may comprise a controlled/living ionic polymerisation method, preferably an anionic polymerisation method.

When the polymeric species is prepared by anionic polymerisation techniques, initiators include, for example, hydrocarbyllithium initiators such as alkyllithium compounds (e.g., methyl lithium, n-butyl lithium, sec-butyl lithium), cycloalkyllithium compounds (e.g., cyclohexyl lithium and aryl lithium compounds (e.g., phenyl lithium, 1-methylstyryl lithium, p-tolyl lithium, naphyl lithium and 1,1-diphenyl-3-methylpentyl lithium. Also, useful initiators include naphthalene sodium, 1,4-disodio-1,1,4,4-tetraphenylbutane, diphenylmethyl potassium or diphenylmethylsodium.

The ionic polymerisation process may be carried out in the absence of moisture and oxygen and in the presence of at least one inert solvent. In one embodiment anionic polymerisation is conducted in the absence of any impurity which is detrimental to an anionic catalyst system. The inert solvent may include a hydrocarbon, an aromatic solvent or ether. Suitable solvents include isobutane, pentane, cyclohexane, benzene, toluene, xylene, tetrahydrofuran, diglyme, tetraglyme, orthoterphenyl, biphenyl, decalin or tetralin,

The ionic polymerisation process may be carried out at a temperature of 0° C. to −78° C.

A more detailed description of process to prepare polymers from an anionic process is discussed in Textbook of Polymer Science, edited by Fred W. Billmeyer Jr., Third Edition, 1984, Chapter 4, pages 88-90.

Step (c) may comprise treating the surface with a polymer or monomers thereof in the presence of a polymerisation initiator, preferably a free radical initiator. The radical initiator may comprise a peroxide. The peroxide may be chosen from: benzoyl peroxide (BPO), di-tert-butyl peroxide, cumene hydroperoxide, tert-butyl hydroperoxide, 2,5-bis(tert-butylperoxy)-2,5-dimethylhexane (DHBP), di(tert-butylperoxyisopropyl)benzene, dicumyl peroxide (DCP), 2,5-di(tert-butylperoxy)-2,5-dimethyl-3-hexyne (DTBPHY) or combinations and/or derivatives thereof. The peroxide may be selected from the group consisting of: benzoyl peroxide (BPO), di-tert-butyl peroxide, cumene hydroperoxide, tert-butyl hydroperoxide, 2,5-bis(tert-butylperoxy)-2,5-dimethylhexane (DHBP), di(tert-butylperoxyisopropyl)benzene, dicumyl peroxide (DCP), 2,5-di(tert-butylperoxy)-2,5-dimethyl-3-hexyne (DTBPHY) or combinations and/or derivatives thereof. The radical initiator may comprise an azo compound. The azo compound may be chosen from: AIBN, AMBN, ADVN, ACVA, dimethyl 2,2′-azobis(2-methylpropionate), AAPH, and 2,2′-azobis[2-(2-imidazolin-2-yl)-propane] dihydrochloride, or combinations and/or derivatives thereof. The azo compound may be selected from the group consisting of: AIBN, AMBN, ADVN, ACVA, dimethyl 2,2′-azobis(2-methylpropionate), AAPH, and 2,2′-azobis[2-(2-imidazolin-2-yl)-propane] dihydrochloride, or combinations and/or derivatives thereof. The radical initiator may comprise a photo-radical initiator, which may be chosen from: camphorquinone, acetophenone, 3-acetophenol, 4-acetophenol, benzophenone, 2-methylbenzophenone, 3-methylbenzophenone, 3-hydroxybenzophenone, 3,4-dimethylbenzophenone, 4-hydroxybenzophenone, 4-benzoylbenzoic acid, 2-benzoylbenzoic acid, methyl 2-benzoylbenzoate, 4,4′-dihydroxybenzophenone, 4-(dimethylamino)-benzophenone, 4,4′-bis(dimethylamino)-benzophenone, 4,4′-bis(diethylamino)-benzophenone, 4,4′-dichlorobenzophenone, 4-(p-tolylthio)benzophenone, 4-phenylbenzophenone, 1,4-dibenzoylbenzene, benzil, 4,4′-dimethylbenzil, p-anisil, 2-benzoyl-2-propanol, 2-hydroxy-4′-(2-hydroxyethoxy)-2-methylpropiophenone (Irgacure 2959), 1-benzoylcyclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio)-2-morpholinopropiophenone, 2-benzyl-2-(dimethylamino)-4′-morpholinobutyrophenone, 2-isonitrosopropiophenone, anthraquinone, 2-ethylanthraquinone, sodium anthraquinone-2-sulfonate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2′-bis(2-chlorophenyl)-4,4′,5,5′-tetraphenyl-1,2′-biimidazole, diphenyl(2,4,6-trimethylbenzoyl)phosphine oxide, phenylbis(2,4,6-trimethylbenzoyl)phosphine oxide, and lithium phenyl (2,4,6-trimethylbenzoyl) phosphinate, or combinations and/or derivatives thereof. The radical initiator may comprise a photo-radical initiator, which may be selected from the group consisting of: camphorquinone, acetophenone, 3-acetophenol, 4-acetophenol, benzophenone, 2-methylbenzophenone, 3-methylbenzophenone, 3-hydroxybenzophenone, 3,4-dimethylbenzophenone, 4-hydroxybenzophenone, 4-benzoylbenzoic acid, 2-benzoylbenzoic acid, methyl 2-benzoylbenzoate, 4,4′-dihydroxybenzophenone, 4-(dimethylamino)-benzophenone, 4,4′-bis(dimethylamino)-benzophenone, 4,4′-bis(diethylamino)-benzophenone, 4,4′-dichlorobenzophenone, 4-(p-tolylthio)benzophenone, 4-phenylbenzophenone, 1,4-dibenzoylbenzene, benzil, 4,4′-dimethylbenzil, p-anisil, 2-benzoyl-2-propanol, 2-hydroxy-4′-(2-hydroxyethoxy)-2-methylpropiophenone (Irgacure 2959), 1-benzoylcyclohexanol, benzoin, anisoin, benzoin methyl ether, benzoin ethyl ether, benzoin isopropyl ether, benzoin isobutyl ether, o-tosylbenzoin, 2,2-diethoxyacetophenone, benzil dimethylketal, 2-methyl-4′-(methylthio)-2-morpholinopropiophenone, 2-benzyl-2-(dimethylamino)-4′-morpholinobutyrophenone, 2-isonitrosopropiophenone, anthraquinone, 2-ethylanthraquinone, sodium anthraquinone-2-sulfonate, 9,10-phenanthrenequinone, 9,10-phenanthrenequinone, dibenzosuberenone, 2-chlorothioxanthone, 2-isopropylthioxanthone, 2,4-diethylthioxanthen-9-one, 2,2′-bis(2-chlorophenyl)-4,4′,5,5′-tetraphenyl-1,2′-biimidazole, diphenyl(2,4,6-trimethylbenzoyl)phosphine oxide, phenylbis(2,4,6-trimethylbenzoyl)phosphine oxide, and lithium phenyl(2,4,6-trimethylbenzoyl phosphinate, or combinations and/or derivatives thereof. The polymerisation initiator may be present in a solution at a total concentration of at least 0.05 wt. %, or at least 0.1, 0.2, 0.3, 0.4, 0.5, 0.6, 0.7, 0.8, 0.9, or at least 1, 1.25, 1.5, 1.75, or at least 2 wt. %. The polymerisation initiator may be present in a solution at a total concentration of no greater than 10 wt. %, or no greater than 9, 8, 7, 6, 5, 4, 3, or no greater than 2 wt. %. The polymerisation initiator may be present in a solution at a total concentration of between 0.1-5 wt. %, or between 0.5-4, 1-3, 1.5-2.5, or between 1.75-2.25 wt. %. In some embodiments, the polymerisation initiator may be present in a solution with the polymer or monomers thereof. The polymerisation initiator may be present in the solution at a greater total concentration than the total concentration of the polymer or monomers thereof. The polymerisation initiator may be present in the solution at a total concentration of at least 1.1 times the total concentration of the polymer or monomers thereof, or at least 1.2, 1.3, 1.4, 1.5, 1.6, 1.7, 1.8, 1.9, or at least 2 times the total concentration of the polymer or monomers thereof.

In some embodiments, the solution of the polymer or monomers thereof comprises a polymerisation initiator and the solution is held for a time period before treating the activated fluoropolymer surface with the solution. The solution may be held for at least 5 minutes prior to treatment, or at least 10, 15, 20, 25, or at least 30 minutes prior to treatment. The solution may be held for no greater than 120 minutes prior to treatment, or no greater than 110, 100, 90, 80, 70, or no greater than 60 minutes prior to treatment. The solution may be held prior to treatment for between 5-85 minutes, 10-80, 20-70, or between 30-60 minutes. The solution may be held prior to treatment at a temperature of at least 5° C. or at least 10, 15 or at least 20° C. The solution may be held prior to treatment at a temperature of no greater than 100° C., or no greater than 90, 80, 70, 60, or no greater than 50° C. The solution may be held prior to treatment at a temperature of between 5-95° C., or between 10-90, 15-85, 20-80, 25-75, 30-70, 35-65, 40-60, or between 45-55° C.

Step (c) may comprise treating the surface with at least one polymer or monomers thereof for a total time of at least 5 minutes, or at least 10, 20, 30, 40, 50, 60, 70, 80, 90, 100, 110, 120, 130, 140, 150, 160, 170, 180, 190, 200, 210, 220, 230, or at least 240 minutes. Step (c) may comprise treating the surface with at least one polymer or monomers thereof for total time of no greater than 10 hours, or no greater than 9, 8, 7, 6, 5, 4.5, or no greater than 4 hours. Step (c) may comprise treating the surface with at least one polymer or monomers thereof for total time of between 0.25-5 hours, or between 0.5-4, or between 0.5-3, or between 0.5-2 hours.

Step (c) may comprise treating the surface with at least one polymer or monomers thereof at a temperature of at least 5° C. or at least 10, 15 or at least 20° C. Step (c) may comprise treating the surface with at least one polymer or monomers thereof at a temperature of no greater than 100° C., or no greater than 90, 80, 70, 60, or no greater than 50° C. Step (c) may comprise treating the surface with at least one polymer or monomers thereof at a temperature of between 5-95° C., or between 10-90, 15-85, 20-80, 25-75, 30-70, 35-65, 40-60, or between 45-55° C. Step (c) may comprise functionalising the surface with at least one glycosaminoglycan, which may be a glycosaminoglycan polymer. At least one glycosaminoglycan may be non-sulphonated. At least one glycosaminoglycan may be anionic, or cationic, or non-ionic. At least one glycosaminoglycan may be anionic. At least one glycosaminoglycan may comprise an anionic non-sulphonated glycosaminoglycan. At least one glycosaminoglycan may have a molecular weight in a range of between about 5000 to about 20,000,000, or from about 10,000 to about 12,000,000, or from about 1,000,000 to about 10,000,000 Da. At least one glycosaminoglycan may be provided in free acid or salt form (glycosaminoglycate). At least one glycosaminoglycate may be associated with any suitable cation, including, but not limited to: alkali metals, such as sodium and potassium; alkaline earth metals; nitrogen-containing cations, such as ammonium, substituted ammonium and quaternized derivatives thereof; and other suitable cations. Preferred salts of Glycosaminoglycan and derivatives thereof include alkali metal or alkaline earth metal glycosaminoglycates. The Glycosaminoglycan may be provided: in pure form; as a mixture of Glycosaminoglycan with proteins and naturally occurring substances derived by the production of Glycosaminoglycan from natural material; or as a chemically modified, Glycosaminoglycan derivative. Mixtures of such glycosaminoglycans may also be provided.

At least one glycosaminoglycan may comprise hyaluronan or a derivative thereof, which may be independently selected from: hylan, heparin, heparan, chondroitin, keratan, dermatan, and sulfates and/or combinations thereof. At least one Glycosaminoglycan may be hyaluronan, or a derivative thereof, which contain repeating disaccharide structure of D-glucuronic acid and 2-acetamido-2-desoxy-D-glucose joined by alternating β1→3 glucuronidic and β1→4 glucosaminidic bonds.

In some embodiments, step (c) comprises functionalising the surface with at least one zwitterionic species, which may comprise a betaine. At least one zwitterionic species may be independently chosen from: a phosphobetaine, a sulfobetaine, a carboxybetaine, and combinations thereof. At least one zwitterionic species may be independently selected from the group consisting of: a phosphobetaine, a sulfobetaine, a carboxybetaine, and combinations thereof.

At least one zwitterionic species may preferably comprise a polymer. At least one zwitterionic species may comprise a polymer containing at least one repeat unit derived from a monomer containing a polymerizable group, preferably an unsaturated group.

At least one polymer may comprise at least one repeat unit derived from a monomer that is independently chosen from: a betaine acrylate, a betaine alkacrylate, a betaine acrylamide, a betaine alkacrylamide, a betaine vinyl compound, a betaine epoxide, and combinations thereof. At least one polymer may comprise at least one repeat unit derived from a monomer that is independently selected from the group consisting of: a betaine acrylate, a betaine alkacrylate, a betaine acrylamide, a betaine alkacrylamide, a betaine vinyl compound, a betaine epoxide, and combinations thereof. At least one polymer may comprise at least one repeat unit derived from a monomer independently chosen from: a betaine acrylate, a betaine alkacrylate, a betaine acrylamide, a betaine alkacrylamide, and combinations thereof. At least one polymer may comprise at least one repeat unit derived from a monomer independently selected from the group consisting of: a betaine acrylate, a betaine alkacrylate, a betaine acrylamide, a betaine alkacrylamide, and combinations thereof. In some embodiments, at least one polymer may comprise at least one repeat unit derived from a monomer independently selected from: a betaine acrylate, a betaine methacrylate, a betaine acrylamide, a betaine methacrylamide, and combinations thereof. In some preferred embodiments, at least one polymer comprises at least one acrylate and/or alkacrylate polymer.

At least one polymer may be a phosphobetaine polymer comprising methacryloyloxyethyl phosphorylcholine (MPC) repeat units.

In some embodiments, step (c) comprises functionalising the surface with at least one chemical species independently chosen from: a glycosaminoglycan, a zwitterionic species, and combinations thereof.

In some embodiments, step (c) comprises functionalising the surface with at least one chemical species independently selected from the group consisting of: a glycosaminoglycan, a zwitterionic species, and combinations thereof.

In some embodiments, step (c) comprises bonding the chemical species to the treated fluoropolymer surface via a linking compound.

In some preferred embodiments, the method of functionalising a fluoropolymer surface of a medical device comprises the steps of:
(a) Providing a medical device comprising a fluoropolymer surface;
(b) Treating the fluoropolymer surface with at least one reducing agent; and
(c) Bonding at least one chemical species to the treated surface via a linking compound to functionalise the treated surface with the at least one chemical species.

In some embodiments, the linking compound comprises a bi- or poly-functional molecule comprising at least two reactive functional groups. A reactive functional group may be independently selected from: a nucleophilic group, an electrophilic group, and a polymerizable moiety. The linking compound may comprise a polymerizable moiety, preferably an unsaturated group, such as a vinyl group. The linking compound may comprise a polymerizable moiety and an electrophilic moiety. The linking compound may comprise a polymerizable unsaturated group, preferably an acrylate or alkacrylate group, such as a methacrylate group. The electrophilic moiety may preferably comprise an electrophilic carbon centre. In some embodiments, the electrophilic carbon centre comprises a carbon atom bonded to an electronegative atom. The carbon atom may be bonded to an electronegative atom independently selected from: a halogen and an oxygen. The electrophilic moiety may comprise an epoxide group. In embodiments, the linking compound comprises glycidyl acrylate and/or a glycidyl alkacrylate. In a particular embodiment, the linking compound is glycidyl methacrylate.

The method may comprise the further step of treating the activated fluoropolymer surface with a linking compound. The method may comprise the step of first bonding the linking compound to the activated fluoropolymer surface, and then bonding at least one chemical species or a monomer thereof to the linking compound. The method may comprise the step of treating the activated surface with the linking compound, optionally in the absence or presence of the species or monomers thereof; and then treating the surface with at least one species or monomers thereof. In some embodiments, the method comprises functionalising the activated surface with the linking compound to form a layer of the linking compound attached to the fluoropolymer surface.

The method may comprise treating the fluoropolymer surface with the linking compound for a total time of at least 5 minutes, or at least 10, 20, 30, 40, 50, or at least 60 minutes. The method may comprise treating the surface with the linking compound for a total time of no greater than 300 minutes, or no greater than 250, 200, or no greater than 150 minutes. The method may comprise treating the surface with the linking compound for a total time of between 20-100 minutes, or between 30-90, 40-80, 50-70, or between 55-65 minutes.

The method may comprise treating the surface with the linking compound at a temperature of at least 5° C. or at least 10, 15 or at least 20° C. The method may comprise treating the surface with the linking compound at a temperature of no greater than 100° C., or no greater than 90, 80, 70, 60, 50, 40, or no greater than 30° C. The method may comprise treating the surface with the linking compound at a temperature of between 5-45° C., or between 10-40, 15-35, or between 20-30° C.

The linking compound may be present neat or as a solution of the linking compound in a solvent. The solvent may be a polar solvent or non-polar solvent. The solvent may be an aprotic solvent. In some embodiments, the solution may be an aqueous solution. Alternatively, the solution may comprise an organic solvent, which may be a polar or non-polar organic solvent. The organic solvent may be independently chosen from: an alcohol, an ether, an ester, a ketone, an aldehyde, an amide, a nitrile, a sulfoxide, a carbonate, a carboxylic acid, and combinations thereof. The organic solvent may be independently selected from the group consisting of: an alcohol, an ether, an ester, a ketone, an aldehyde, an amide, a nitrile, a sulfoxide, a carbonate, a carboxylic acid, and combinations thereof. In some embodiments, the solvent is or comprises an ether, which may be a C1-C20 ether, preferably C1-C10 ether. The ether may be an alkyl tert-butyl ether, which may be independently chosen from: methyl tert-butyl ether, ethyl tert-butyl ether, propyl tert-butyl ether, and combinations thereof. The ether may be an alkyl tert-butyl ether, which may be independently selected from the group consisting of: methyl tert-butyl ether, ethyl tert-butyl ether, propyl tert-butyl ether, and combinations thereof. The linking compound may be present in the solution at a total concentration of at least 0.05 wt. %, or at least 0.1, 0.2, 0.3, 0.4, 0.5, 0.6, 0.7, 0.8, 0.9, 1, 1.5, or at least 2 wt. %. The linking compound may be present in the solution at a total concentration of no greater than 10 wt. %, or no greater than 9, 8, 7, 6, 5, 4, 3, or no greater than 2 wt. %. The linking compound may be present in the solution at a total concentration of between 0.05-10 wt %, or between 0.1-5 wt. %, or between 0.5-4, or between 1-3, or between 1.5-2.5 wt %.

In some embodiments, steps (b) and (c) are performed simultaneously. In other embodiments, step (c) may be performed subsequently to step (b).

In some preferred embodiments, the method of functionalising a fluoropolymer surface of a medical device comprises in order the steps of:
(a) Providing a medical device comprising a fluoropolymer surface;
(b) Treating the fluoropolymer surface with at least one reducing agent; and then
(c) Functionalising the treated surface with at least one chemical species.

In some embodiments, step (b) is performed in the presence of at least one chemical species. Where the chemical species comprises a polymer, step (b) may be performed in the presence of at least one monomer thereof. In other embodiments, step (b) is performed in the absence of the chemical species or monomer thereof, preferably prior to addition of the chemical species or monomer thereof.

In embodiments in which at least one species is bonded to the fluoropolymer surface through a linking compound, the method may comprise the step of functionalising the fluoropolymer surface with the linking compound simultaneously or subsequently to step (b). In some embodiments, the activation step is performed in the presence of the linking compound. In other embodiments, the activation step is performed in the absence of the linking compound, preferably prior to addition of the linking compound.

The method of the second aspect of the invention may further comprise a step of sonicating the fluoropolymer surface, as described for the first aspect of the invention. The sonication step may be performed at one or more of the following times: after step (b), at the end of step (c), and any combination thereof.

The method of the second aspect of the invention may further comprise a step of washing the fluoropolymer surface, as described for the first aspect of the invention. The washing step may be performed at one or more of the following times: after step (b), at the end of step (c), and any combination thereof.

According to a third aspect of the invention, there is provided a medical device comprising an activated fluoropolymer surface obtainable by a method comprising the steps of:
(a) Providing a medical device comprising a fluoropolymer surface; and
(b) Treating the fluoropolymer surface with at least one reducing agent.

The medical device is preferably obtainable by the method of the first aspect of the inventions. Statements of invention above relating to any previous aspect of the invention may also be applied mutatis mutandis to the third aspect of the invention.

According to the fourth aspect of the invention, there is provided an infusion set or patch pump comprising a cannula comprising an activated fluoropolymer surface obtainable by a method comprising the steps of:
(a) Providing a cannula comprising a fluoropolymer surface; and
(b) Treating the fluoropolymer surface with at least one reducing agent.

The cannula may preferably be a medical device of the third aspect of the invention. The cannula is preferably obtainable by a method of the first aspect of the invention. Statements of invention above relating to any previous aspect of the invention may also be applied mutatis mutandis to the fourth aspect of the invention.

According to a fifth aspect of the invention, there is provided a method of delivering a substance or removing a substance from the body of a subject, the method comprising the steps of:
(a) Inserting a medical device of the third aspect of the invention into the body; and
(b) Delivering a substance to or removing a substance from the body via the medical device.

The medical device may be a catheter or cannula, preferably as described for the first aspect of the invention. The medical device may be a cannula that is part of an infusion set or patch pump, as for the fourth aspect of the invention above.

The method may comprise inserting the medical device into the body intravenously and/or subcutaneously.

The substance may be a drug. In some embodiments, the substance comprises insulin. Step (b) of the method may comprise delivering insulin to the body via the medical device.

Statements of invention above relating to any previous aspect of the invention may also be applied mutatis mutandis to the fifth aspect of the invention.

### DETAILED DESCRIPTION OF THE INVENTION

In order that the invention may be more clearly understood embodiments thereof will now be described, by way of example only, with reference to the accompanying drawings, of which:

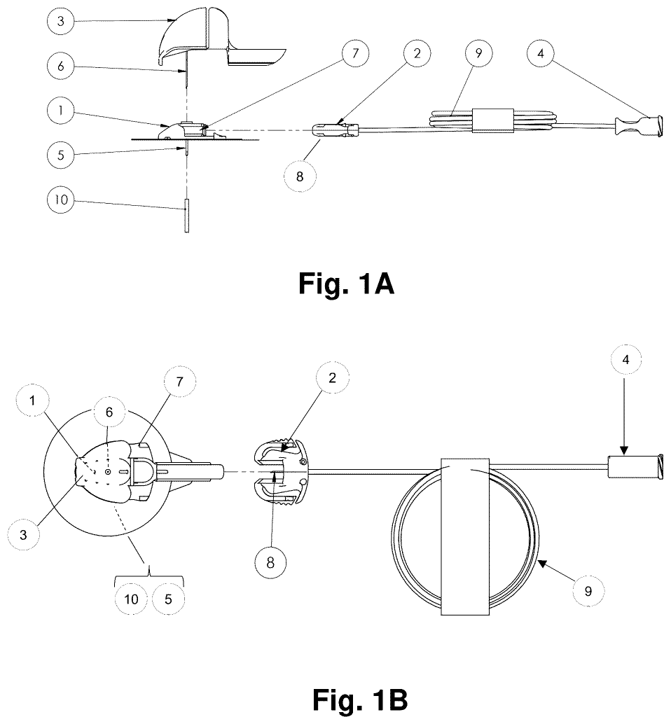

FIGS. 1A-1B shows an exploded side-on view (FIG. 1A); and a top-down view of an infusion set of the fourth aspect of the invention (FIG. 1B). Dashed lines represent points of connection of the components of the infusion set.

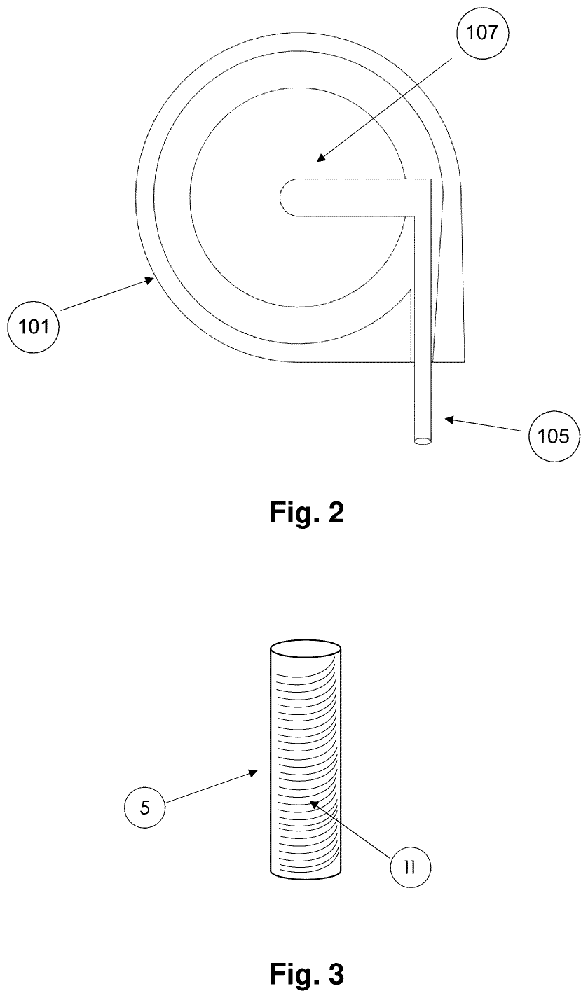

FIG. 2 shows a cross-sectional view of a patch pump of the fourth aspect of the invention.

FIG. 3 shows an expanded side-on view of the cannula (5) as displayed in FIG. 1A.

### INFUSION SET

An embodiment of a medical device of the invention having an activated fluoropolymer surface was prepared.

The cannula was part of an infusion set of the fourth aspect of the invention for the subcutaneous delivery of insulin. Diagrams of the infusion set are displayed in FIGS. 1A and 1B. With reference to the figures, the infusion set comprises a body (1), which is attachable to the skin of a user via an adhesive part of the body (1). The infusion set comprises the cannula (5) which extends from and projects away from the body (1) of the infusion set in the same direction that the adhesive part of the body (1) faces.

The body (1) comprises a fluid part (7), which is part of the body and provides a fluid path through the infusion set, allowing for fluid communication between the body (1) and the cannula (5). The fluid part (7) also contains a cartridge of insulin (not shown) for subcutaneous delivery. The arrangement also allows for fluid communication between the inside of the insulin cartridge and the cannula (5). The fluid part (7) is connected to a pump (not shown) via tubing (9). The fluid part (7) is connected to the tubing (9) at one end thereof via a connector needle (8) of a set connector (2). The other end of the tubing (9) contains a pump connector (4) through which the tubing (9) is attached to the pump.

The fluid part (7) and body (1) contain a channel extending therethrough which is aligned with the cannula (5). Such an arrangement allows for an insertion needle (6) to be passed through the channel and into the cannula (5), with the insertion needle (6) projecting in the same direction as the cannula (5) and extending out of the free, distal end of the cannula (5). An inserter (3) is connected to the insertion needle (6) and the needle (6) extends from the inserter (3). The infusion set further includes a needle cover (10) in which the insertion needle (6) is sheathed before use.

In use, the body (1) of the infusion set is attached to the skin of the user via the adhesive part of the body (1). The free end of the cannula (5), which projects from the body (1), is inserted into the body of the user with assistance from the insertion needle (6), which is inserted using the inserter (3) through the channel extending through the fluid part (7) and body (1) and through the cannula (5). The insertion needle (6) contacts the skin of the user and is inserted into the body of the user before the cannula (5), making insertion of the cannula (5) easier.

On insertion of the cannula (5) into the body of the user, the fluid part (7) is connected to the pump as described above. The insulin is delivered subcutaneously from the infusion set via the cannula (5), with assistance from the pump.

### PATCH PUMP

A further embodiment of a medical device of the invention having an activated fluoropolymer surface was prepared in the same manner as for the infusion set above.

The cannula is part of a patch pump of the fourth aspect of the invention for the subcutaneous delivery of insulin. A cross-sectional view of the patch pump is displayed in FIG. 2. With reference to FIG. 2, the patch pump comprises a body (101), which is attachable to the skin of a user via an adhesive part of the body (101). The patch pump comprises a cannula (105) which extends from the body (101).

The body (101) comprises a fluid part (107), which is part of the body and provides a fluid path through the patch pump, allowing for fluid communication between the body (101) and the cannula (105). The fluid part (107) also contains a cartridge of insulin (not shown) for subcutaneous delivery. The arrangement also allows for fluid communication between the inside of the insulin cartridge and the cannula (105).

The body (101) further comprises an inbuilt pump (not shown).

In use, the body (101) of the patch pump is attached to the skin of the user via the adhesive part of the body (101). The free end of the cannula (105), which projects from the body (101), is inserted into the body of the user. Insulin is delivered subcutaneously from the patch pump via the cannula (105), with assistance from the inbuilt pump.

### Activation of Medical Device Fluoropolymer Surface

The above medical devices having activated fluoropolymer surfaces were prepared by a method of the invention as follows:

A cannula containing a polymeric tubular body having a PTFE outer surface was provided.

A solution of sodium naphthalide in diglyme was preheated to 60° C. for 1 hour. The solution was thereafter agitated for 2-3 seconds, after which the cannula was submerged in the solution for 30 seconds. The cannula was then removed and immediately rinsed with isopropyl alcohol for 10 seconds. The cannula was then further rinsed with 70° C. deionised water for 15 seconds. The cannula was then left to air dry overnight.

XPS data for the prepared activated surface showed that the fluorine-to-carbon ratio had changed from 2:1 (before surface activation) to 1:6 (after surface activation). XPS data also showed the presence of C—O bonds on the treated surface.

### Results

A protein adsorption test was performed to assess the impact of treating the fluoropolymer surface of the cannula with a reducing agent on the protein adsorption behaviour of the PTFE surface. The fluoropolymer surface of the cannula was treated with a bovine serum albumin (BSA) protein solution. BSA adsorption was assessed by fluorescence after 24- and 72-hours treatment.

The cannula which had been treated with the reducing agent demonstrated reduced fluorescence after both 24 and 72 hours compared to an untreated cannula control, which displayed substantial fluorescence after both time periods. This demonstrated that treating the fluoropolymer surface with a reducing agent resulted in reduced protein adsorption on the surface.

These results highlight an inherent protein-repellent behaviour of fluoropolymer surfaces of medical devices which have been activated by treatment with a reducing agent.

Further, the mechanical properties of the PTFE cannula were not negatively impacted, and the PTFE retained its lubricious non-stick surface.

### Functionalisation of the Activated Fluoropolymer Surface

The activated fluoropolymer surface of the medical device was then functionalised with a phosphobetaine polymer as follows:

The cannula was submerged in a solution of glycidyl methacrylate (2 wt % in methyl tert-butyl ether) at room temperature for 1 hour. The cannula was then removed, rinsed with deionised water at ambient temperature, and sonicated for 10 minutes in fresh deionised water. The cannula was then air dried.

A solution containing 0.5 wt % of methacryloyloxyethyl phosphorylcholine (MPC) and 1 wt % of an AIBN polymerisation initiator was prepared in a methyl tert-butyl ether solvent. The prepared solution was held at 50° C. for 30-60 minutes prior to use to initiate polymerisation. The cannula was submerged in the prepared solution at 50° C. for 1 hour, prior to rinsing with water, sonicating and air drying, as performed previously. Submerging the cannula in the solution allowed for covalent linkage of phosphobetaine polymer chains to a linker derived from glycidyl methacrylate which was present on the fluoropolymer surface.

### Results

The functionalised cannula contained a thin layer of phosphobetaine polymer adsorbed to the fluoropolymer surface via a linker.

This is illustrated in FIG. 3, which shows an expanded side-on view of the cannula (5) of the infusion set, as displayed in FIG. 1A. FIG. 3 displays a layer or coating (11) of phosphobetaine polymer which is adsorbed to the fluoropolymer surface.

Similar attempted functionalisation of a cannula containing a non-treated fluoropolymer surface failed to generate such a phosphobetaine layer.

A protein adsorption test was performed as described above. The functionalised cannula demonstrated minimal fluorescence after both 24 and 72 hours of treatment, which suggested minimal protein adsorption had occurred on the functionalised surface.

Again, the mechanical properties of the PTFE cannula were not negatively impacted, and the PTFE retained its lubricious non-stick surface.

The above embodiments are described by way of example only. Many variations are possible without departing from the scope of the invention as defined in the appended claims.

1. A method of activating a fluoropolymer surface of a medical device, the method comprising the steps of:
a. Providing a medical device comprising a fluoropolymer surface; and
b. Treating the fluoropolymer surface with at least one reducing agent.

2. A method as claimed in claim 1, wherein the fluoropolymer is independently chosen from: polytetrafluoroethylene, polyvinylfluoride, polyvinylidene fluoride, polychlorotrifluoroethylene, a perfluoroalkoxy polymer, fluorinated ethylene-propylene, polyethylenetetrafluoroethylene, polyethylenechlorotrifluoroethylene, a perfluoroelastomer, a fluoroelastomer, perfluoropolyether, perfluorosulfonic acid, perfluoropolyoxetane, and combinations, blends or copolymers thereof, and wherein the fluoropolymer preferably comprises polytetrafluoroethylene.

3. A method as claimed in any claim 1, wherein the medical device comprises a tubular body comprising the fluoropolymer surface.

4. A method as claimed in claim 3, wherein the fluoropolymer surface comprises an outer surface of the tubular body, and preferably comprises at least 70% of the outer surface area of the tubular body.

5. (canceled)

6. A method as claimed in claim 1, wherein the medical device is a cannula that is part of an infusion set or patch pump.

7. A method as claimed in claim 1, wherein the at least one reducing agent is independently chosen from: an alkali metal, an alkaline earth metal, a group III metal, a transition metal, and combinations thereof, and wherein the at least one reducing agent preferably comprises an alkali metal that is independently chosen from: lithium, potassium, sodium, and combinations thereof.

8. A method as claimed in claim 1, wherein the at least one reducing agent is provided as a solution of the at least one reducing agent in a carrier solvent, wherein the carrier solvent preferably comprises an aprotic ether, preferably an aprotic glycol ether, wherein step (b) comprises submerging the medical device in the solution comprising the at least one reducing agent.

9. (canceled)

10. A method as claimed in claim 1, wherein step (b) comprises treating the fluoropolymer surface with the at least one reducing agent at a temperature of between 45-65° C., or between 50-65° C.

11. A method as claimed in claim 1, wherein step (b) comprises treating the fluoropolymer surface with the at least one reducing agent for between 5-55 seconds.

12. A method as claimed in claim 1, wherein step (b) comprises activating the fluoropolymer surface across at least 70% of the total area of the fluoropolymer surface.

13. A method as claimed in claim 1, wherein step (b) comprises defluorinating or partially defluorinating the fluoropolymer surface.

14. A method as claimed in claim 1, wherein step (b) comprises reducing the average fluorine-to-carbon atomic ratio of the fluoropolymer surface to a value of no greater than 1.2.

15. A method as claimed in claim 1, wherein step (b) comprises increasing the average surface energy of the fluoropolymer surface to a value of at least 25 mN/m.

16. A method as claimed in claim 1, wherein step (b) comprises reducing the average contact angle of the fluoropolymer surface to a value of no greater than 80°.

17. A method as claimed in claim 1, wherein the method further comprises the step of treating the fluoropolymer surface with ultraviolet light.

18. A method as claimed in claim 1, wherein step (b) comprises reducing the average fluorine-to-carbon atomic ratio of the fluoropolymer surface to a value of no greater than 0.3, preferably no greater than 0.2.

19. A method as claimed in claim 26, wherein step (c) comprises applying the at least one chemical species to the treated surface as a coating comprising the at least one chemical species, wherein step (b) comprises reducing the fluoropolymer surface and then oxidising the surface; and step (c) comprises functionalising the oxidised surface with the at least one chemical species, wherein the at least one chemical species comprises a polymer.

20. (canceled)

21. (canceled)

22. A method as claimed in claim 19, wherein step (b) comprises creating at least one polymerisation initiation site on the surface; and step (c) comprises polymerising a monomer on at least one polymerisation initiation site to functionalise the surface with at least one polymer.

23. (canceled)

24. (canceled)

25. A method as claimed in any one of claims 26, wherein step (c) comprises bonding the chemical species to the surface via a linking compound.

26. A method of functionalising a fluoropolymer surface of a medical device, the method comprising the steps of:
a. Providing a medical device comprising a fluoropolymer surface;
b. Treating the fluoropolymer surface with at least one reducing agent; and
c. Functionalising the treated surface with at least one chemical species.
